(19) United States
(12) Patent Application Publication
(10) Pub. No.: US 2021/0340545 A1
(43) Pub. Date: Nov. 4, 2021

(54) METHODS AND COMPOSITIONS FOR PREVENTION AND TREATMENT OF RESPIRATORY INFECTIONS

(71) Applicant: UpRNA, LLC, Cambridge, MA (US)

(72) Inventor: David DEAMER, Santa Cruz, CA (US)

(21) Appl. No.: 17/306,515

(22) Filed: May 3, 2021

Related U.S. Application Data
(60) Provisional application No. 63/019,590, filed on May 4, 2020.

(51) Int. Cl. C12N 15/113; A61K 9/127; A61K 31/685; A61K 31/683; A61K 31/045; A61K 31/09; A61K 47/12; A61K 31/7105; A61K 9/00

(52) U.S. Cl. CPC C12N 15/1131; A61K 9/127; A61K 31/685; A61K 31/683; C12N 2310/14; A61K 31/09; A61K 47/12; A61K 31/7105; A61K 9/0073; A61K 31/045

(57) ABSTRACT

This technology relates in part to methods and compositions for treatment or prevention of infections, including but not limited to respiratory infections. The methods and compositions can include lung surfactants, detergents, antivirals, siRNA, and nicotinamide adenine dinucleotide (NAD) boosting agents.

### CROSS-REFERENCE TO RELATED APPLICATIONS

This application claims priority to U.S. Provisional Patent Application no. 63/019,590, filed on May 4, 2020, entitled METHODS AND COMPOSITIONS FOR PREVENTION AND TREATMENT OF RESPIRATORY INFECTIONS, naming David Deamer as inventor, and designated by attorney docket number UPR-1001-PV, the contents of which are incorporated herein by reference in their entirety.

### FIELD

The technology relates to compositions that include surfactants or detergents for treating or preventing respiratory infections, and methods of prevention or treatment using the same.

### BACKGROUND

Disease-causing pathogens have the potential to affect many areas in the body, including the reproductive, respiratory, and gastrointestinal systems. They also can affect the liver, brain, and skin. Some pathogens, such as viruses, have been implicated in a variety of cancers. Certain viral infections can be contagious, such as those caused by the common cold, influenza, chickenpox, human immunodeficiency virus (HIV), SARS-CoV-2 and other viruses. Some of these infections, such as COVID-19 caused by SARS-CoV-2, are highly contagious and can be deadly, resulting in a world pandemic crisis. Hence, methods of prevention and/or treatment of pathogenic infections are much needed.

### SUMMARY

Infectious diseases, such as those caused by viruses, can be treated or prevented by a variety of methods, individually or in combination.

For infectious agents with at least partially known genome sequences, small interfering RNA (siRNA) can be prepared that targets specific sequences in the genome. If targeted siRNA can be delivered, e.g., to lung epithelial cells, it can bind to RNA-induced silencing complexes (RISC). When viral RNA enters a cell, the RISC-siRNA complex can bind to and degrade or disable viral RNA that is involved in viral mechanisms, such as synthesis of capsid proteins and the assembly of new virus particles.

Additionally, other agents can be delivered alone or in combination. Non-limiting examples of agents include artificial lung surfactants or other detergents, and antimicrobial agents (e.g., antivirals, antibacterials).

Additionally, nicotinamide adenine dinucleotide (NAD) boosting agents can be delivered, alone or in combination. In addition to generally boosting function, increasing NAD levels can help prevent COVID-19 or other infectious diseases, and can improve outcomes for COVID-19 or other infectious diseases once contracted, including sequelae thereof.

There can be a synergistic effect by delivering agents together or in close succession, for example targeted siRNA with other agents such as detergents and surfactants. Without being limited by theory, synergistic effects include but are not limited to: (1) as surfactant spreads over the surfaces of alveolar epithelial cells, it can conceal receptors required for the infectious agent (e.g., viral particles to bind); (2) the components of an infectious agent (e.g., virus, bacterium) are held together by non-covalent bonds (e.g., viral capsid, bacterial membrane), and detergents such as soaps (e.g., fatty acids such as myristic acid) can disrupt and inactivate the infectious agent; and (3) free siRNA in the mixture can be taken up by the lung epithelial cells and be available in the cytoplasm.

Agents can be delivered within liposomes. For example, siRNA, antimicrobial agents, or combinations thereof can be delivered within liposomes. When the lipid membrane is digested by lipases associated with normal lipid metabolism, the siRNA will be released, thereby providing another path for intracellular siRNA to be available in the cytoplasm. The liposomes containing encapsulated siRNA can also be taken into the cells by endocytosis. Similarly, liposome digestion and/or uptake can aid in delivery of other agents, such as antimicrobial agents.

Delivery can be as an aerosol to be inhaled by subjects.

Subjects can include health workers, other essential workers, or other people who are exposed to patients exhibiting symptoms of an infection (e.g., COVID-19). It can also be delivered to individuals who have early symptoms related to a possible infection.

The agents and compositions provided herein can be administered for the treatment or prevention of diseases and conditions in a subject. Thus, provided herein are methods of preventing or treating a disease or condition in a subject by administering one or more of the agents or compositions provided herein. In aspects, a disease or condition is associated with a virus. In aspects, a disease or condition is a respiratory infection. In certain aspects, the respiratory infection is caused by or associated with a virus. In aspects, the virus is SARS-CoV-2. In aspects, the disease or condition is COVID-19.

As used herein, treatment means any manner in which a condition, disorder or disease, or symptoms thereof, are ameliorated or otherwise beneficially altered. For example, the disorder or disease, or symptoms thereof, are ameliorated or otherwise beneficially altered as manifested by an improvement of about or at 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more, up to 100% relative to the health of the subject prior to administering the treatment. Hence, treatment encompasses prophylaxis, therapy and/or cure. Treatment also encompasses any pharmaceutical use of the compositions provided herein.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, prevention or prophylaxis refers to methods in which the risk of developing a disease or condition is reduced. Prophylaxis includes reduction in the risk of developing a disease or condition and/or a prevention of worsening of symptoms or progression of a disease or reduction in the risk of worsening of symptoms or progression of a disease. For example, the risk of developing a disease or condition and/or a prevention of worsening of symptoms or progression of a disease or reduction in the risk of worsening of symptoms can be reduced by about or at 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more, up to 100% relative to the risk prior to administering the preventative.

### DETAILED DESCRIPTION

All publications, patents, and patent applications mentioned in this specification are herein incorporated by reference to the same extent as if each individual publication, patent, or patent application was specifically and individually indicated to be incorporated by reference.

### Detergents and Surfactants

Surfactants or detergents can be employed to treat or prevent respiratory infection, including but not limited to COVID-19. Soaps, fatty acids, and other detergents or surfactants can be delivered to a subject with or at risk of a respiratory infection. The components of many infectious agents (e.g., virus, bacterium) are held together by non-covalent bonds (e.g., viral capsid, bacterial membrane), and detergents such as soaps (e.g., fatty acids such as myristic acid) can disrupt and inactivate the infectious agent.

Detergents and surfactants can be chosen, at least in part, based on melting point. For example, detergent and surfactants can be used that have melting points close to, around, or below body temperature (e.g., lung temperature). This can help ensure the detergents and surfactants are not solid after administration and are able to be distributed. In some cases, detergents and surfactants are chosen that have a melting point less than or equal to 100° C., 95° C., 90° C., 85° C., 80° C., 75° C., 70 ° C., 65° C., 60° C., 55° C., 50° C., 45° C., 40° C., 37° C., 35° C., 30° C., 25° C., or 20° C. Detergents and surfactant melting points can be assessed for the compound on its own, or for the compound in the context of the overall composition being administered; a detergent or surfactant with a higher melting point on its own may still be suitable for use as the overall melting point of a mixed composition can be lower.

Detergents and surfactants can be chosen, at least in part, based on molecular charge and/or pKa. For example, detergents and surfactants can be chosen that will be anionic at neutral pH or at physiological (e.g., lung) pH.

Non-limiting examples of detergents include fatty acids and derivatives thereof, including but not limited to salts thereof. As for other detergents, fatty acid detergents can be selected based on their melting point. Saturated, mono-unsaturated, and poly-unsaturated fatty acid detergents can all be employed. In some cases, saturated fatty acid detergents are C12 to C16 fatty acids. In some cases, unsaturated fatty acid detergents are C12 to C18 or C12 to C20 fatty acids. Non-limiting examples of fatty acids include capric acid, undecylic acid, lauric acid, tridecylic acid, myristic acid, pentadecylic acid, palmitic acid, margaric acid, stearic acid, nonadecylic acid, and arachidic acid (010:0 to C20:0), C10:1-020:1 fatty acids, C10:2-020:2 fatty acids, 010:3-020:3 fatty acids, and others.

Mixtures of suitable detergents and surfactants are also contemplated for use herein.

### Artificial Lung Surfactants

A surfactant utilized sometimes is an artificial lung surfactant. Artificial lung surfactants have been employed as treatment for infantile respiratory distress syndrome (IRDS). By supplying natural and/or replacement lung surfactants to subjects with insufficient native surfactant levels (such as premature infants), alveoli can be allowed to expand, and lung function can be improved.

Artificial lung surfactants can be administered to subjects with or at risk of respiratory infection, including but not limited to COVID-19. The lung surfactant can spread over the surfaces of the lungs, such as the alveolar epithelium. The presence of the lung surfactant can open up lung passageways and improve disease symptoms such as pneumonia. The lung surfactant can also disrupt the activity of an infectious agent, for example by concealing receptors to which the infectious agent would bind or by disrupting the infectious agent itself (e.g., detergent disruption of a viral capsid).

Artificial lung surfactants can also aid in delivery of other treatments, such as co-administered drugs and other compounds. As the lung surfactant coats the lung surfaces, it can help convey other treatments, such as those discussed herein. siRNA

Small interfering RNA (siRNA), also known as short interfering RNA or silencing RNA, is a class of RNA molecules capable of repressing translation of mRNA. Without being limited by theory, siRNA can be incorporated with proteins to form an RNA-Induced Silencing Complex (RISC). The RISC can then bind to target mRNA that is complementary to the siRNA and cut it, causing degradation of the mRNA and preventing it from being translated into protein.

The activity of an infectious agent can be disrupted at a variety of points along its pathway. For example, a virus can be disrupted at the synthesis of new copies of its genome (e.g., by silencing expression of viral polymerases or by silencing copies of viral RNA itself), at the production of viral proteins such as capsid proteins (e.g., by silencing expression of those proteins), or at the insertion into the host genome for retroviruses (e.g., by silencing expression of reverse transcriptases and/or integrases).

SARS-CoV-2, for example, can be disrupted by silencing of the viral genome itself (e.g., upon release into host cells) and/or by repression of translation for proteins including but not limited to the spike protein (e.g., at the receptor-binding domain (RBD)), polymerases (e.g., RNA-dependent RNA polymerase (RdRp)), proteases (e.g., coronavirus main protease (3CLpro), papain-like protease (PLpro)), envelope proteins, or capsid or nucleocapsid proteins (ACS Cent. Sci. 2020, 6, 3, 315-331, Publication Date: Mar. 12, 2020, DOI: 10.1021/acscentsci.0c00272).

In some cases, the siRNA can target highly conserved regions, allowing the siRNA to remain effective across various strains and mutants of the pathogen. Non-limiting examples of highly conserved regions include regions encoding enzyme active sites or receptor binding domains.

### Antimicrobial Agents

One or more antimicrobial agents sometimes are utilized, non-limiting examples of which include antiviral agents, antibacterial agents and antifungal agents.

Non-limiting examples of antiviral agents include neuramidase inhibitors (including but not limited to oseltamivir, zanamivir, peramivir), endonuclease inhibitors (e.g., cap-dependent endonuclease inhibitors) (including but not limited to baloxavir marboxil), adamantanes (including but not limited to amantadine, rimantadine), virus-associated protein (VAP) anti-idiotypic antibodies, viral receptor ligands, anti-receptor antibodies, anti-VAP antibodies, receptor anti-idiotypic antibodies, extraneous receptor mimics, synthetic receptor mimics, entry inhibitors (including but not limited to enfuvirtide), uncoating inhibitors (including but not limited to adamantadines, pleconaril), nucleotide or nucleoside analogs or other compounds that deactivate viral enzymes or terminate nucleotide synthesis (including but not limited to acyclovir, zidovudine, lamivudine, remdesivir), integrase inhibitors, transcription inhibitors, translation inhibitors (e.g., antisense molecules including morpholino antisense oligonucleotides and/or ribozyme antivirals; including but not limited to fomivirsen), protein processing inhibitors, protease inhibitors, double-stranded RNA activated caspase oligomerizer (DRACO), assembly inhibitors (including but not limited to rifampicin), and release inhibitors (including but not limited to zanamivir, oseltamivir).

Non-limiting examples of antibacterial agents include agents targeting the bacterial cell wall (including but not limited to penicillins and cephalosporins), agents targeting the cell membrane (including but not limited to polymyxins), and agents targeting essential bacterial enzymes (including but not limited to rifamycins, lipiarmycins, quinolones, and sulfonamides), cyclic lipopeptides (including but not limited to daptomycin), glycylcyclines (including but not limited to tigecycline), oxazolidinones (including but not limited to linezolid), and lipiarmycins (including but not limited to fidaxomicin).

Non-limiting examples of antifungal agents include polyenes (including but not limited to amphotericin b, candicidin, filipin, hamycin, natamycin, nystatin, rimocidin), azoles such as imidazoles, triazoles, and thiazoles (including but not limited to bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, posaconazole, propiconazole, ravuconazole, terconazole, voriconazole, abafungin), echinocandins (including but not limited to anidulafungin, caspofungin, micafungin), and other antifungals (including but not limited to aurones, benzoic acid, ciclopirox, flucytosine or 5-fluorocytosine, griseofulvin, haloprogin, tolnaftate, undecylenic acid, triacetin, crystal violet, castellani's paint, orotomide, miltefosine, potassium iodide, nikkomycin, coal tar, copper(ii) sulfate, selenium disulfide, sodium thiosulfate, piroctone olamine, iodoquinol(diiodohydroxyquin), clioquinol, acrisorcin, zinc pyrithione, sulfur).

### NAD Boosters

One or more nicotinamide adenine dinucleotide (NAD) boosting compounds, such as nicotinamide mononucleotide (NMN), salts, or prodrugs thereof, sometimes are utilized.

Nicotinamide mononucleotide (NMN) is produced from nicotinamide in the nicotinamide adenine dinucleotide (NAD) biosynthesis pathway, a reaction that is catalyzed by Nampt. NMN is further converted to NAD in the NAD biosynthesis pathway, a reaction that is catalyzed by Nmnat. NAD is produced from the conversion of nicotinamide to NMN, which is catalyzed by Nampt, and the subsequent conversion of NMN to NAD, which is catalyzed by Nmnat. In mammals, the functional homolog of yeast PNC1 is NAMPT, which also catalyzes the first step in NAD salvage. NAMPT catalyzes the formation of nicotinamide mononucleotide (NMN) from NAM, which is then converted to NAD by NMNAT1, NMNAT2, and NMNAT3. Nicotinamide riboside, a precursor to NAD, enters the salvage pathway after being converted to NMN by nicotinamide riboside kinase (NRK) enzymes.

Thus, diseases, disorders and conditions that are affected by NAD levels are likewise affected by the amount of NMN precursor available for NAD biosynthesis, and thus can be treated by administering the NMN compounds and compositions disclosed herein.

NMN can work through the nicotinamide mononucleotide adenylyltransferase (Nmnatl) pathway or other pathways of NAD biosynthesis which have nutritional and/or therapeutic value in improving plasma lipid profiles, prevention of stroke, and/or prolonging life and well-being. Other embodiments relate to a method for preventing or treating a disease or condition associated with the nicotinamide mononucleotide adenylyltransferase (Nmnatl) pathway or other pathways of NAD biosynthesis by administering a composition comprising NMN. Diseases or conditions which typically have altered levels of NAD or its precursors which can be prevented or treated by supplementing a diet or therapeutic treatment regime with NMN and/or NAD+include, but are not limited to, lipid disorders, (e.g., dyslipidemia, hypercholesterolaemia or hyperlipidemia), stroke, type I and II diabetes, cardiovascular disease, and other physical problems associated with obesity.

In addition to generally boosting function, increasing NAD levels can help prevent COVID-19 or other infectious diseases, and can improve outcomes for COVID-19 or other infectious diseases once contracted, including sequelae thereof. See, for example, Miller, R et al. “COVID-19: NAD+deficiency may predispose the aged, obese and type2 diabetics to mortality through its effect on SIRT1 activity.” Medical hypotheses vol. 144 (2020): 110044. doi:10.1016/j.mehy.2020.110044; Omran, HM and Almaliki, MS, “Influence of NAD+as an ageing-related immunomodulator on COVID 19 infection: A hypothesis.” Journal of Infection and Public Health vol. 13 issue 9 (Sept. 2020) pp. 1196-1201. doi: 10.1016/j.jiph.2020.06.004; Jiang, Y. et al. “Treatment of SARS-CoV-2 induced pneumonia with NAD+in a mouse model.” Research Square preprint (Oct. 2020). doi:10.21203/rs.3.rs-96999/v1.

### Liposomes

Liposomes are spherical vesicles comprising at least one lipid bilayer, including but not limited to phospholipids (e.g., phosphatidylcholine). Liposomes can include ligands, which can aid in targeting liposome delivery (e.g., to diseased tissue). Liposomes include but are not limited to multilamellar vesicles (MLVs), small unilamellar liposome vesicles (SUVs), large unilamellar liposome vesicles (LUVs), and cochleate vesicles. Liposomes can be generated by a variety of methods, including but not limited to disruption of membranes (e.g., by sonication).

Liposomes can be used for drug delivery. For example, liposomes can encapsulate or otherwise incorporate drugs and deliver them to cells. Liposomes can deliver drugs to cells by merging with cell membranes, or by opening near cells or other targets.

Lipsomes can contain one or more different agents or compositions including those disclosed herein, in the liposome membrane, within the liposome, or both.

### Delivery

A composition described herein can be administered to a subject in need thereof (e.g., a disease patient, a person at risk of disease) individually or in combination. In some cases, as discussed herein, there are benefits to administration of multiple compositions together or in short succession.

A composition described herein can be administered via inhalation (e.g., aerosol). A composition described herein can be administered as a coating on an instrument; for example, an intubation tube can be coated in compositions prior to intubation of a subject.

A composition described herein can be administered before intubation, during intubation, or after intubation of a subject.

### Non-limiting Embodiments

Listed hereafter are non-limiting examples of certain embodiments of the technology.

A1. A composition for treating or preventing a respiratory infection in a subject, comprising a surfactant.

A2. The composition of embodiment A1, wherein the surfactant comprises a fatty acid.

A3. The composition of embodiment A2, wherein the fatty acid is a C10-C18 fatty acid.

The composition of embodiment A2 or A3, wherein the fatty acid is a saturated fatty acid.

A5. The composition of embodiment A2 or A3, wherein the fatty acid is a monounsaturated fatty acid.

A6. The composition of embodiment A2 or A3, wherein the fatty acid is a polyunsaturated fatty acid.

A7. The composition of embodiment A2, wherein the fatty acid is myristic acid.

A8. The composition of embodiment A2, wherein the fatty acid is palmitic acid.

A9. The composition of any one of embodiments A1 to A8, further comprising small interfering RNA (siRNA).

A10. The composition of embodiment A9, wherein the siRNA targets a nucleic acid of an infectious agent.

A11. The composition of embodiment A10, wherein the nucleic acid is an RNA.

Al2. The composition of embodiment A11, wherein the RNA is a messenger RNA (mRNA).

A13. The composition of any one of embodiments A1 to A12, wherein the infectious agent associated with the respiratory infection is a virus.

A14. The composition of embodiment A13, wherein the virus is a coronavirus.

A15. The composition of embodiment A14, wherein the coronavirus is SARS-CoV-2.

A16. The composition of any one of embodiments A1 to A15, comprising a nicotinamide adenine dinucleotide (NAD) booster.

A17. The composition of embodiment A16, wherein the NAD booster comprises nicotinamide mononucleotide (NMN) or a salt thereof.

A18. The composition of embodiment A16, wherein the NAD booster comprises nicotinamide mononucleotide (NMN), a prodrug thereof, or a salt thereof.

A19. The composition of any one of embodiments A16 to A18, wherein the surfactant comprises the NAD booster.

A20. The composition of any one of embodiments A1 to A19 that is formulated for prophylactic administration.

A21. The composition of any one of embodiments A1 to A20 that is formulated as a liposome.

A22. The composition of any one of embodiments A1 to A21 that is formulated as an aerosol.

A23. The composition of any one of embodiments A1 to A22, wherein the subject is a patient.

A24. The composition of any one of embodiments A1 to A22, wherein the subject is a health care worker.

B1. A composition for treating or preventing a respiratory infection in a subject, comprising small interfering RNA (siRNA).

B2. The composition of embodiment B1, wherein the siRNA targets a nucleic acid of an infectious agent.

B3. The composition of embodiment B2, wherein the nucleic acid is an RNA.

B4. The composition of embodiment B3, wherein the RNA is a messenger RNA (mRNA).

B5. The composition of any one of embodiments B1 to B4, wherein the infectious agent associated with the respiratory infection is a virus.

B6. The composition of embodiment B5, wherein the virus is a coronavirus.

B7. The composition of embodiment B6, wherein the coronavirus is SARS-CoV-2.

B8. The composition of any one of embodiments B1 to B7, further comprising a surfactant or a detergent.

B9. The composition of embodiment B8, comprising a fatty acid.

B10. The composition of embodiment B9, wherein the fatty acid is a C10-C18 fatty acid.

B11. The composition of embodiment B8 or B9, wherein the fatty acid is a saturated fatty acid.

B12. The composition of embodiment B8 or B9, wherein the fatty acid is a monounsaturated fatty acid.

B13. The composition of embodiment B8 or B9, wherein the fatty acid is a polyunsaturated fatty acid.

B14. The composition of embodiment B9, wherein the fatty acid is myristic acid.

B15. The composition of embodiment B9, wherein the fatty acid is palmitic acid.

B16. The composition of any one of embodiments B1 to B15, comprising a nicotinamide adenine dinucleotide (NAD) booster.

B17. The composition of embodiment B16, wherein the NAD booster comprises nicotinamide mononucleotide (NMN) or a salt thereof.

B18. The composition of embodiment B16, wherein the NAD booster comprises nicotinamide mononucleotide (NMN), a prodrug thereof, or a salt thereof.

B19. The composition of any one of embodiments B16 to B18, wherein the surfactant comprises the NAD booster.

B20. The composition of any one of embodiments B1 to B19 that is formulated for prophylactic administration.

B21. The composition of any one of embodiments B1 to B20 that is formulated as a liposome.

B22. The composition of any one of embodiments B1 to B21 that is formulated as an aerosol.

B23. The composition of any one of embodiments B1 to B22, wherein the subject is a patient.

B24. The composition of any one of embodiments B1 to B22, wherein the subject is a health care worker.

C1. A composition for treating or preventing a respiratory infection in a subject, comprising liposomes comprising drugs.

C2. The composition of embodiment C1, wherein the drugs comprise wherein the drugs comprise small interfering RNA (siRNA).

C3. The composition of embodiment C1 or C2, wherein the drugs comprise antivirals.

C4. The composition of embodiment C2 or C3, wherein the siRNA targets a nucleic acid of an infectious agent.

C5. The composition of embodiment C4, wherein the nucleic acid is an RNA.

C6. The composition of embodiment C5, wherein the RNA is a messenger RNA (mRNA).

C7. The composition of any one of embodiments C1 to C4, wherein the infectious agent associated with the respiratory infection is a virus.

C8. The composition of embodiment C7, wherein the virus is a coronavirus.

C9. The composition of embodiment C8, wherein the coronavirus is SARS-CoV-2.

C10. The composition of any one of embodiments C1 to C9, comprising a nicotinamide adenine dinucleotide (NAD) booster.

C11. The composition of embodiment C10, wherein the NAD booster comprises nicotinamide mononucleotide (NMN) or a salt thereof.

C12. The composition of embodiment C10, wherein the NAD booster comprises nicotinamide mononucleotide (NMN), a prodrug thereof, or a salt thereof.

C13. The composition of any one of embodiments C1 to C12, further comprising a surfactant or a detergent.

C14. The composition of embodiment C13, wherein the surfactant or detergent comprises a NAD booster.

C15. The composition of any one of embodiments C1 to C14 that is formulated for prophylactic administration.

C16. The composition of any one of embodiments C1 to C15 that is formulated as an aerosol.

C17. The composition of any one of embodiments C1 to C16, wherein the subject is a patient.

C18. The composition of any one of embodiments C1 to C16, wherein the subject is a health care worker.

D1. A composition for treating or preventing a respiratory infection in a subject, comprising one or more pulmonary surfactants.

D2. The composition of embodiment D1, wherein the one or more pulmonary surfactants comprises dipalmitoylphosphatidylcholine (DPPC).

D3. The composition of embodiment D1 or D2, wherein the one or more pulmonary surfactants comprises phosphatidylglycerol (PG).

D4. The composition of any one of embodiments D1 to D3, wherein the one or more pulmonary surfactants comprises hexadecanol.

D5. The composition of any one of embodiments D1 to D4, wherein the one or more pulmonary surfactants comprises tyloxapol.

D6. The composition of any one of embodiments D1 to D5, further comprising small interfering RNA (siRNA).

D7. The composition of embodiment D6, wherein the siRNA targets a nucleic acid of an infectious agent.

D8. The composition of embodiment D7, wherein the nucleic acid is an RNA.

D9. The composition of embodiment D8, wherein the RNA is a messenger RNA (mRNA).

D10. The composition of any one of embodiments D1 to D9, wherein the infectious agent associated with the respiratory infection is a virus.

D11. The composition of embodiment D10, wherein the virus is a coronavirus.

D12. The composition of embodiment D11, wherein the coronavirus is SARS-CoV-2.

D13. The composition of any one of embodiments D1 to D12, comprising a nicotinamide adenine dinucleotide (NAD) booster.

D14. The composition of embodiment D13, wherein the NAD booster comprises nicotinamide mononucleotide (NMN) or a salt thereof.

D15. The composition of embodiment D13, wherein the NAD booster comprises nicotinamide mononucleotide (NMN), a prodrug thereof, or a salt thereof.

D16. The composition of embodiment any one of embodiments D1 to D15, wherein the pulmonary surfactant comprises the NAD booster.

D17. The composition of any one of embodiments D1 to D16 that is formulated for prophylactic administration.

D18. The composition of any one of embodiments D1 to D17 that is formulated as an aerosol.

D19. The composition of any one of embodiments D1 to D18 that is formulated as a liposome.

D20. The composition of any one of embodiments D1 to D19, wherein the subject is a patient.

D21. The composition of any one of embodiments D1 to D20, wherein the subject is a health care worker.

E1. A composition, comprising:
(a) a pulmonary surfactant;
(b) a fatty acid;
(c) a liposome; and
(d) small interfering RNA (siRNA).

E2. The composition of embodiment E1, wherein the liposome comprises one or more antivirals.

E3. The composition of embodiment E1 or E2, wherein the liposome comprises the siRNA.

The composition of any one of embodiments E1 to E3, wherein the siRNA targets a nucleic acid of an infectious agent.

E5. The composition of embodiment E4, wherein the nucleic acid is an RNA.

E6. The composition of embodiment E5, wherein the RNA is a messenger RNA (mRNA).

E7. The composition of any one of embodiments E1 to E6 that is formulated for prevention or treatment of a respiratory infection associated with an infectious agent.

E8. The composition of any one of embodiments E4 to E7, wherein the infectious agent is a virus.

E9. The composition of embodiment E8, wherein the virus is a coronavirus.

E10. The composition of embodiment E9, wherein the coronavirus is SARS-CoV-2.

E11. The composition of any one of embodiments E1 to E10, wherein the pulmonary surfactant comprises dipalmitoylphosphatidylcholine (DPPC).

E12. The composition of any one of embodiments E1 to E11, wherein the pulmonary surfactant comprises phosphatidylglycerol (PG).

E13. The composition of any one of embodiments E1 to E12, wherein the pulmonary surfactant comprises hexadecanol.

E14. The composition of any one of embodiments E1 to E13, wherein the pulmonary surfactant comprises tyloxapol.

E15. The composition of any one of embodiments E1 to E14, wherein the fatty acid is a C10-C18 fatty acid.

E16. The composition of any one of embodiments E1 to E14, wherein the fatty acid is a saturated fatty acid.

E17. The composition of any one of embodiments E1 to E14, wherein the fatty acid is a monounsaturated fatty acid.

E18. The composition of any one of embodiments E1 to E14, wherein the fatty acid is a polyunsaturated fatty acid.

E19. The composition of any one of embodiments E1 to E14, wherein the fatty acid is myristic acid.

E20. The composition of any one of embodiments E1 to E14, wherein the fatty acid is palmitic acid.

E21. The composition of any one of embodiments E1 to E20 that is formulated for prophylactic administration.

E22. The composition of any one of embodiments E1 to E20 that is formulated for administration as an aerosol.

E23. The composition of any one of embodiments E1 to E22, comprising a nicotinamide adenine dinucleotide (NAD) booster.

E24. The composition of embodiment E23, wherein the NAD booster comprises nicotinamide mononucleotide (NMN) or a salt thereof.

E25. The composition of embodiment E23, wherein the NAD booster comprises nicotinamide mononucleotide (NMN), a prodrug thereof, or a salt thereof.

E26. The composition of any one of embodiments E23 to E25, wherein the pulmonary surfactant comprises the NAD booster.

F1. A pharmaceutical composition, comprising: the composition of any one of embodiments A1 to A24, B1 to B24, C1 to C18, D1 to

D21 and E1 to E26; and a physiologically acceptable carrier or excipient.

G1. A method of treating or preventing a respiratory infection, comprising administering to a subject in need thereof the composition of any one of embodiments A1 to A24, B1 to

B24, C1 to C18, D1 to D21 and E1 to E26, or the pharmaceutical composition of embodiment F1, in an amount that is effective for treatment or prevention of the respiratory infection.

G2. The method of embodiment G1, wherein the infectious agent associated with the respiratory infection is a virus.

G3. The method of embodiment G2, wherein the virus is a coronavirus.

G4. The method of embodiment G3, wherein the coronavirus is SARS-CoV-2.

G5. The method of any one of embodiments G1 to G4, wherein the subject is a patient.

G6. The method of any one of embodiments G1 to G4, wherein the subject is a health care worker.

H1. A method of treating or preventing a respiratory infection, comprising: administering to a subject a composition comprising one or more of:
i. a pulmonary surfactant;
ii. a fatty acid;
iii. liposomes; and
iv. small interfering RNA (siRNA).

H2. The method of embodiment H1, wherein the liposomes comprise antivirals.

H3. The method of embodiment H1 or H2, wherein the liposomes comprise the siRNA.

H4. The method of any one of embodiments H1 to H3, wherein the siRNA targets a nucleic acid of an infectious agent.

H5. The method of embodiment H4, wherein the infectious agent is a virus.

H6. The method of embodiment H5, wherein the virus is a coronavirus.

H7. The method of embodiment H6, wherein the coronavirus is SARS-CoV-2.

H8. The method of any one of embodiments H1 to H7, wherein the pulmonary surfactant comprises dipalmitoylphosphatidylcholine (DPPC).

H9. The method of any one of embodiments H1 to H8, wherein the pulmonary surfactant comprises phosphatidylglycerol (PG).

H10. The method of any one of embodiments H1 to H9, wherein the pulmonary surfactant comprises hexadecanol.

H11. The method of any one of embodiments H1 to H10, wherein the pulmonary surfactant comprises tyloxapol.

H12. The method of any one of embodiments H1 to H11, wherein the fatty acid is a C10-C18 fatty acid.

H13. The method of any one of embodiments H1 to H11, wherein the fatty acid is a saturated fatty acid.

H14. The method of any one of embodiments H1 to H11, wherein the fatty acid is a monounsaturated fatty acid.

H15. The method of any one of embodiments H1 to H11, wherein the fatty acid is a polyunsaturated fatty acid.

H16. The method of any one of embodiments H1 to H11, wherein the fatty acid is myristic acid.

H17. The method of any one of embodiments H1 to H11, wherein the fatty acid is palmitic acid.

H18. The method of any one of embodiments H1 to H17, wherein the subject is a patient.

H19. The method of any one of embodiments H1 to H18, wherein the subject is a health care worker.

H20. The method of any one of embodiments H1 to H19, wherein the composition is administered prophylactically.

H21. The method of any one of embodiments H1 to H20, wherein the composition is administered in an aerosol.

H22. The method of any one of embodiments H1 to H21, wherein the composition comprises a nicotinamide adenine dinucleotide (NAD) booster.

H23. The method of embodiment H22, wherein the NAD booster comprises nicotinamide mononucleotide (NMN) or a salt thereof.

H24. The method of embodiment H22, wherein the NAD booster comprises nicotinamide mononucleotide (NMN), a prodrug thereof, or a salt thereof.

H25. The method of any one of embodiments H1 to H24, wherein the pulmonary surfactant comprises the NAD booster.

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology has been described with reference to specific implementations. The terms and expressions that have been utilized herein to describe the technology are descriptive and not necessarily limiting. The terms and expressions that have been employed are used as terms of description and not of limitation and use of such terms and expressions do not exclude any equivalents of the features shown and described or portions thereof, and various modifications are possible within the scope of the technology claimed. Thus, it should be understood that although the present technology has been specifically disclosed by representative embodiments and optional features, modification and variation of the concepts herein disclosed may be resorted to by those skilled in the art, and such modifications and variations are considered within the scope of this technology.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

We claim:

1. A composition, comprising:
(a) a pulmonary surfactant;
(b) a fatty acid;
(c) a liposome; and
(d) small interfering RNA (siRNA).

2. The composition of claim 1, wherein the liposome comprises one or more antivirals.

3. The composition of claim 1, wherein the liposome comprises the siRNA.

4. The composition of claim 1, wherein the siRNA targets a nucleic acid of an infectious agent.

5. The composition of claim 4, wherein the nucleic acid is an RNA.

6. The composition of claim 5, wherein the RNA is a messenger RNA (mRNA).

7. The composition of claim 1 that is formulated for prevention or treatment of a respiratory infection associated with an infectious agent.

8. The composition of claim 7, wherein the infectious agent is a virus.

9. The composition of claim 8, wherein the virus is a coronavirus.

10. The composition of claim 9, wherein the coronavirus is SARS-CoV-2.

11. The composition of claim 1, wherein the pulmonary surfactant comprises dipalmitoylphosphatidylcholine (DPPC).

12. The composition of claim 1, wherein the pulmonary surfactant comprises phosphatidylglycerol (PG).

13. The composition of claim 1, wherein the pulmonary surfactant comprises hexadecanol.

14. The composition of claim 1, wherein the pulmonary surfactant comprises tyloxapol.

15. The composition of claim 1, wherein the fatty acid is a C10-C18 fatty acid.

16. The composition of claim 1, wherein the fatty acid is a saturated fatty acid.

17. The composition of claim 1, wherein the fatty acid is a monounsaturated fatty acid.

18. The composition of claim 1, wherein the fatty acid is a polyunsaturated fatty acid.

19. The composition of claim 1, wherein the fatty acid is myristic acid.

20. The composition of claim 1, wherein the fatty acid is palmitic acid.

21. The composition of claim 1 that is formulated for prophylactic administration.

22. The composition of claim 1 that is formulated for administration as an aerosol.

23. The composition of claim 1, comprising a nicotinamide adenine dinucleotide (NAD) booster.

24. The composition of claim 23, wherein the NAD booster comprises nicotinamide mononucleotide (NMN) or a salt thereof.

25. The composition of claim 23, wherein the NAD booster comprises nicotinamide mononucleotide (NMN), a prodrug thereof, or a salt thereof.

26. The composition of claim 23, wherein the pulmonary surfactant comprises the NAD booster.

27. A method of treating or preventing a respiratory infection, comprising administering to a subject in need thereof a composition comprising:
(a) a pulmonary surfactant;
(b) a fatty acid;
(c) a liposome; and
(d) small interfering RNA (siRNA),
in an amount that is effective for treatment or prevention of the respiratory infection.
